(19) United States
(12) Patent Application Publication
(10) Pub. No.: US 2026/0191788 A1
(43) Pub. Date: Jul. 9, 2026

(54) MANUFACTURING OF A PHARMACEUTICAL DOSAGE FORM WITH 3D MELT DOSING

(71) Applicant: Merck Patent GMBH, Darmstadt (DE)

(72) Inventors: Thomas Kipping, Darmstadt (DE); Nabil Lamrabet, Darmstadt (DE)

(21) Appl. No.: 19/135,231

(22) PCT Filed: Dec. 12, 2023
(86) PCT No.: PCT/EP2023/085270

§ 371 Date: Jun. 3, 2025

(30) Foreign Application Priority Data
Dec. 15, 2022 (EP) 22213687.1

(51) Int. Cl. A61K 9/20; A61K 9/14; A61K 31/496; A61K 31/522

(52) U.S. Cl. CPC A61K 9/2095; A61K 9/146; A61K 9/2031; A61K 31/496; A61K 31/522

(57) ABSTRACT

The invention relates to the use of a polymer for the manufacturing of a pharmaceutical dosage form with 3D melt dosing and a process for manufacturing of a pharmaceutical dosage form with 3D melt dosing using a polymer including the pharmaceutical dosage form obtainable by such a process.

### TECHNICAL FIELD

The invention relates to the use of a polymer for the manufacturing of a pharmaceutical dosage form with 3D melt dosing and a process for manufacturing of a pharmaceutical dosage form with 3D melt dosing using a polymer including the pharmaceutical dosage form obtainable by such a process.

### BACKGROUND

Creating individual and personalized medications for individual patients can be very complex. With standard tableting processes for each variation of concentration a new formulation needs to be developed and the pre-mixture needs to be adapted. This process can be very complex and time consuming. The conventional pharmaceutical manufacturing is based on mass production of selected dosage strengths. This can create challenges especially looking at chronic diseases or taking into account different subgroups of patients such as paediatric or geriatric populations.

Therefore there is a high need to look into alternative manufacturing technologies that are providing a higher flexibility for creating the final dosage form.

Looking at oral drug delivery in the framework of personalized medicine it can be observed that an increasing knowledge of personalized medicine also requires the need for individual dosing. Drug dosage forms are urgently needed enabling an individual therapy, especially for oral drug delivery. Oral drug delivery is still the most important and most frequently used application route. Therefore, suitable oral dosage forms with the option for individualized dosing are urgently needed. Several different technologies are evaluated to provide an individualized dosing concept. One common practice is the splitting of tablets and breakdown into smaller subunits. This maybe an effective solution to provide a rapid dose adaptation, but bears the risk of missdosing caused by deviations of mass. Therefore there is a high need to enable reliable individual dosing of oral forms.

Additive manufacturing is another important technology to create dosage forms of versatile dosing strengths. As one additive manufacturing technology, 3D melt dosing provides the advantage of individual dosing of multiple ingredients like drug substances or polymeric carriers. The individual melt can be deposited directly in predefined molds. As the deposition device can freely move in all different directions, a precise dosing of material can be achieved within the form. The dosing can also be performed in pre-defined blisters that can be directly sealed after the filling process by and directly create the final primary packaging. Currently, the technology is predominantly applied in decoration lines, where depositors with CAD (computer aided design) can move in all different dimensions and therefore can create a high level of material deposition. Also the creation of several layers during one production step is possible with this system.

The modification of release rates of a pharmaceutical dosage form, manufactured with 3D melt dosing, can be challenging as the system is not able to create defined structures. As soon as the melt solidifies in the moulds the material usually forms dense structures with rather low porositie. The strong structure does not allow the modification of release rates, as it would be required to enable personalized medication.

Another problem during the manufacturing process is the appearance of “edging” leading to a concave surface is created on top of the molds. As a consequence the surface finishing is negatively impacted by an uneven geometry which can be problematic for the final dose due to mechanical stability and optical appearance.

### SUMMARY OF THE INVENTION

It has been found that polymers with a melting point between 20° C. and 200° C. are particularly suitable for the manufacturing of a pharmaceutical dosage form with 3D melt dosing.

In another aspect, the invention provides a process for manufacturing of a pharmaceutical dosage form with 3D melt dosing, comprising the steps of melting a polymer with a melting point between 20° C. and 200° C., adding at least one active pharmaceutical ingredient to the molden polymer and dispensing the mixture through a first nozzle into a mould. The outer shape of the mould is pre-defined by the geometry of the form and can be adjusted to various sizes or geometries.

In one embodiment, the polymer as mentioned above is used together with a plasticizer. Surprisingly, the addition of plasticizers enabled the modification of release rates and/or improved the surface finishing of the pharmaceutical dosage forms.

In a further embodiment of the invention, the polymer is a low-melting polymer with a melting point between 20° C. and 100° C. In a particular embodiment, the polymer is PEG 6000 or Poloxamer 188.

In a further embodiment of the invention, the polymer further comprises a plasticizer.

In one embodiment of the invention, a multiple dosing systems is used. In a two nozzle system for example the amount of drug containing polymer can be easily modified by filling the remaining parts with the drug-free polymer from another nozzle. In this way even for high dose variations the same shape and weight of tablets can be created.

The process allows for the manufacturing of a pharmaceutical dosage form with the same geometry but different final drug contents and allows a simple variation of dosage form creation during the entire manufacturing process. Multiple layer tablets can be created as well as different layer systems by utilizing a one-shot or multiple-shot technology. By the use of the CAD-based deposition technology also defined areas of melt can be deposited within one mould enabling the generation of complex multi-drug systems.

The process can also be used for directly dosing of the melt into blisters and sealing them to create the final product in the respective primary packaging.

### DETAILED DESCRIPTION OF THE INVENTION

An embodiment of the invention is a process for manufacturing of a pharmaceutical dosage form with 3D melt dosing, comprising the steps of (i) melting a polymer with a melting point between 20° C. and 200° C., (ii) adding at least one active pharmaceutical ingredient to the molden polymer and (iii) dispensing the mixture through a first nozzle into a mould.

According to the invention the term “3D melt dosing” is a process in which a melt of polymer and drug substance is generated and and then dosed with a high accuracy in pre-defined moulds. This process can be performed via a one-shot system or eben combine different melt forms by using multi-shot distributors. In this case several layers of material can be created.

The first step of a preferred manufacturing process is transferring the melt from the beaker to the machine vessel. The tray which contains the moulding form or the blisters is put onto a running conveyor in the right position. The conveyor transports the tray further until it reaches the filling sensor, so that the tray stops precisely under the nozzle. Meanwhile, the nozzle moves to its initial position. During this step, the pumps fill with a specified amount of volume. The nozzle lower itself to the target position (near the ground of the form/blister) and the piston turns and start to discharge the melt. The nozzle remains in this position durning the wait delay to enable a complete discharge. After that, the nozzle moves vertically upwards while the piston continue to discharge the melt. After discharging the melt, the piston rotate to the suction position and the nozzle ascend back to initial position. The conveyor moves the tray to the next mould after the fill pause is expired. The suction cycle of the piston is also performed simultaneously. The right distance between each form can be adjusted with the distance centering button.

This work procedure repeats itself until all desired number of forms are filled. The tray moves then to the stop sensor and the production process is finished.

In a preferred embodiment a second polymer with or without a polymer is simultaneously or sequentially dispensed through the same nozzle into the mould. This configuration makes it possible for the pharmaceutical dosage form to be produced in a single shot, also called the one-shot system, leading to pharmaceutical dosage form with a shell and a core in which the pharmaceutical substance is contained. A simple form of the one-shot process for food is described, for example, in WO 94/02027. It is known in confectionery machine construction for the production of pralines in which chocolate for the outer shell is poured through a nozzle into a cavity and the contents of the praline are filled into this forming chocolate shell with another nozzle, so that a closed praline is finally produced. According to the present invention, this one-shot system provides for a polymer supply line in a nozzle which is arranged around the supply line for the polymer and API in the same nozzle. This means that the outlets of both supply lines are arranged in or above the cavity, so that now only the addition of the substances for the shell and the addition of the pharmaceutical substance has to be controlled.

In a further preferred embodiment more than two polymers, with or without API, can be used.

In a further preferred embodiment a second polymer is simultaneously or sequentially dispensed through a second nozzle into the mould. This is an alternative system of the 3D melt dosing machine using multiple nozzles for the dosing of the polymer melts with or without API (multiple-shot system).

In an extended application, the melt is not prepared outside the 3D melt dosing machine and placed into a machine vessel but placed into the 3D melt dosing machine in form of cartridges. The cadriges contain the polymers with or without API. Depending on the desired composition of the pharmaceutical dosage form, different cartridges can be combined to produce the pharmaceutical dosage form. Catriges are equipped with heating devices that can preferably be controlled individually. 3D melt dosing machines equipped with a cartridge system are particularly suitable for higher melting polymer, e.g. above 80° C. or 100° C.

In a further extended application also depositors with CAD-execution can freely move the depositor-head in all directions and therefore provide a high flexibility in the detailed composition of the final tablet. Different melt systems can be distributed within one tablet.

In a further embodiment, different depositors are used to create multilayer tablets.

To enable the process of individual dosing suitable polymers needed to be identified. It was found that polymers having a melting point between 20° C. and 200° C. are suitable candidates for this technology. According to the present invention suitable polymers are polymers that have a melting point between 20° C. and 200° C.

Many amorphous polymers do not crystallize under normal conditions and are considered as amorphous. Amorphous polymers have no detailed melting point but but their amorphous regions undergo an important phase transition the so called glass transition temperature. This temperature is linked to the conjugated main chain motion of the polymer. This is linked to a softening of the polymer and the effect can be detected as a step in the baseline of a differential scanning calormetry measurement.

Crystalline or semi-crystalline polymers show also a more defined melting point. The melting point is determined as the temperature at which a polymer transits from the crystalline state into the viscous flow state. Melting endotherms can be detected via thermal characterization like for example via differential scanning calorimetry.

In a preferred embodiment, polymers of the present invention are low-melting polymer having a melting point between 20° C. and 100° C., more preferably between 20° C. and 80° C.

According to the invention suitable polymers are poloxamers, polyethylene glycols or polyvinyl alcohols having a melting point between 20° C. and 200° C., preferably poloxamers and polyethylene glycols having a melting point between 20° C. and 200° C.

In a preferred embodiment of the invention, the polymer is a crystalline or semi-crystalline polymer. Crystalline polymers: The polymer is aligning in crystalline structures and provides a defined melting point in thermograms.

Semi-crystalline polymers beneath their crystalline structures still maintain a high amount of amorphous regions. In this cases both a Tg and a Tm can be detected in thermograms.

Poloxamers are amphiphilic polymers, with two hydrophilic blocks and a hydrophobic block in the middle. A poloxamer is a polyethylene glycol (PEG)/polypropylene glycol (PPG) tri-block copolymer whereby one PPG block is flanked on both sides with a PEG block. The polyethylene glycol (PEG) part is often also called polyethylene oxide (PEO) part. The polypropylene glycol (PPG) part is often also called the polypropylene oxide (PPO) part.

Poloxamer grades are commonly named with the letter P (for poloxamer) followed by three digits that is officially used by USP and EP. It describes the composition of the polymer as follows: the first two digits multiplied by 100 represents the molecular weight of the PO block and the last digit multiplied by 10 provides the percentage of EO in %.

Poloxamer P188 in average is composed of 80% EO, while the remaining 20% PO make up for 1800 g/mol. Poloxamer P407 is a poloxamer with an average polyoxypropylene molecular mass of 4000 g/mol and a 70% polyoxyethylene content.

Poloxamers have the General Formula (I)

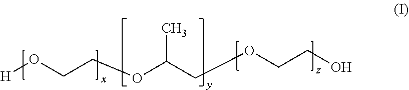

For different poloxamers numbers of x (PEO), y (PPO chain) and z (PEO) are varying over a broad range, depending on the type of poloxamer. For poloxamer P188 the PPO chain contains in the average a unit number ranging from 25 to 30, and each PEO is composed of 75 to 85 EO units in average, with a molecular weight ranging from 7680 to 9510 Da. For poloxamer P407 the PPO chain contains in the average a unit number of 56, and each PEO is composed of approximately 101 EO units in average, with an molecular weight ranging from 9840 to 14600 Da.

Poloxamer 407 (a=101, b=56) with molecular weight ranging from 9840 to 14600 Da.

Table 1 is showing types of poloxamers monographed in the European Pharmacopoeia (Ph. Eur.) and United States Pharmacopeia (USP).

TABLE 1

| | Average | | | |
|---|---|---|---|---|
| | Molecular | | EO units (each | PO units Ph. |
| | Weight | Weight EO | flank Ph. Eur. | Eur. (range); |
| Poloxamer | (USP/EP) | [%] USP | (range); USP | USP |
| 124 | 2090-2360 | 46.7 ± 1.9 | 10-15; 12 | 18-23; 20 |
| 188 | 7680-9510 | 81.8 ± 1.9 | 75-85; 80 | 25-30; 27 |
| 237 | 6840-8830 | 72.4 ± 1.9 | 60-68; 64 | 35-40; 37 |
| 338 | 12,700-17,400 | 83.1 ± 1.7 | 137-146; 141 | 42-47; 44 |
| 407 | 9840-14,600 | 73.2 ± 1.7 | 95-105; 101 | 54-60; 56 |

Table 2 is showing the melting point, molecular weight and weight percentage of ethylene oxide chains (Weight EO %) of the most common poloxamers as published in Russo, Villa. Poloxamer Hydrogels for Biomedical Applications. Pharmaceutics. 2019; 11:671.

TABLE 2

| | Weight | Average Molecular | Melting |
|---|---|---|---|
| Poloxamer | EO [%] | Weight [Da] | Point [° C.] |
| P105 | 50 | 1900 | 7 |
| P108 | 80 | 4700 | 48 |
| P122 | 20 | 1630 | −26 |
| P123 | 30 | 1850 | −1 |
| P124 | 40 | 2200 | 16 |
| P182 | 20 | 2500 | −4 |
| P183 | 30 | 2650 | 10 |
| P184 | 40 | 2900 | 16 |
| P185 | 50 | 3400 | 27 |
| P188 | 80 | 8400 | 52 |
| P212 | 20 | 2750 | −7 |
| P215 | 50 | 4150 | 27 |
| P217 | 70 | 6600 | 48 |
| P234 | 40 | 4200 | 34 |
| P235 | 50 | 4600 | 34 |
| P237 | 70 | 7700 | 49 |
| P238 | 80 | 11,400 | 54 |
| P288 | 80 | 13,000 | 58 |
| P333 | 30 | 4950 | 30 |
| P334 | 40 | 5900 | 32 |
| P335 | 50 | 6500 | 35 |
| P338 | 80 | 14,600 | 57 |
| P402 | 20 | 5000 | 20 |
| P403 | 30 | 5750 | 31 |
| P407 | 70 | 12,600 | 56 |

Some poloxamers are commercially available at different ratios of EO and PO units and in different forms like liquids, pastes and wax-like solids, e.g. Synperonic® (Croda International PLC), Pluronic® (BASF SE), Lutrol® (BASF SE renamed in Kolliphor® and Kollisolv®) or Poloxamer 188 EMPROVER EXPERT.

Alternatively, poloxamers can be made from raw materials according to methods known in the art (see, for example, U.S. Pat. Nos. 3,579,465 and 3,740,421).

Further information about poloxamers can be found in Hagers Handbuch der Pharmazeutischen Praxis, volume 9 “Stoffe P-Z”, 1994, pages 282 to 284 or Russo, Villa. Poloxamer Hydrogels for Biomedical Applications. Pharmaceutics. 2019; 11:671.

The use of specific poloxamer grades according to the invention is of interest for the formulation of solid oral pharmaceutical dosage forms with an instant, immediate or prolonged API release. An instant or immediate release is preferred.

Poloxamers according to the invention have a melting point of 20° C. or higher.

In a further embodiment, poloxamers according to the invention have a melting point between 20° C. and 60° C., between 30° C. and 60° C., between 40° C. and 60° C. or between 50° C. and 60° C. In a further embodiment, poloxamers according to the invention have an average molecular weight of 3000 Da or higher, between 4000 and 15000 Da or between 7000 and 13000 Da. In a further embodiment, poloxamers according to the invention have a weight percentage of ethylene oxide chains between 50% and 90% or 70% to 85%. In another embodiment the poloxamer is poloxamer P188 or P407.

In a further embodiment, poloxamers according to the invention have a melting point between 20° C. and 60° C., an average molecular weight of 3000 Da or higher and a weight percentage of ethylene oxide chains between 50% and 90%.

In a further embodiment, poloxamers according to the invention have a melting point between 30° C. and 60° C. an average molecular weight between 4000 and 15000 Da and a weight percentage of ethylene oxide chains between 70% to 85%.

In a further embodiment, poloxamers according to the invention have a melting point between 50° C. and 60° C. an average molecular weight between 7000 and 13000 Da and a weight percentage of ethylene oxide chains between 70% to 85%.

In a further embodiment, poloxamers according to the invention have a melting point between 20° C. and 55° C., between 30° C. and 55° C., between 40° C. and 55° C. or between 50° C. and 55° C. In a further embodiment, poloxamers according to the invention have an average molecular weight between 4000 and 12000 Da or between 7000 and 12000 Da. In a further embodiment, poloxamers according to the invention have a weight percentage of ethylene oxide chains between 75% and 85% or 80% to 85%. In another embodiment the poloxamer is poloxamer P188.

Polyethylene glycol (PEG) is a polyether compound derived from petroleum also known as polyethylene oxide (PEO) or polyoxyethylene (POE). The structure of PEG is commonly expressed as H—(O—CH₂—CH₂)ₙ—OH. In general, the notation “PEG MW” is used where MW is the molecular weight. PEG is widely used in the pharmaceutical industry as a drug carrier. It is a water-soluble polymer that is nonflammable, nontoxic, and biodegradable and thereby also harmless to the environment.

The polyethylene glycol (PEG) according to the present invention has an average molecular weight, in Daltons, within the range 1000 to 35000, preferably PEG 1000, PEG 2000, PEG 3000, PEG 4000, PEG 5000, PEG 10000, PEG 20000, PEG 35000, in particular PEG 6000. Polyethylene glycols are defined and identified according to the European Pharmacopoeia 11.0 described in the Monograph “Macrogols”.

Polyvinyl alcohol (PVA) is a synthetic water-soluble polymer that has the idealized formula [CH₂CH(OH)]ₙ. It possesses good film-forming, adhesive, and emulsifying properties. PVA is prepared from polyvinyl acetate, where the functional acetate groups are either partially or completely hydrolysed to alcohol functional groups. If not completely hydrolysed, PVA is a random copolymer consisting of vinyl alcohol repeat units —[CH₂CH(OH)]— and vinyl acetate repeat units —[CH₂CH(OOCCH₃)]—. The polarity of PVA is closely linked to its molecular structure. The hydrolysis degree and the molecular weight determine the molecular properties of PVA. As the degree of hydrolysis of acetate groups increases, the solubility of the polymer in aqueous media and also crystallinity and melting temperature of the polymer increase. However, at high hydrolysis degrees over 88%, the solubility of PVA decreases again. PVA is generally soluble in water, but almost insoluble in almost all organic solvents, excluding, in some cases, ethanol.

The typical PVA nomenclature indicates the viscosity of a 4% solution at 20° C. and the degree of hydrolysis of the polymer. For example, PVA 3-83 is a PVA grade with a viscosity of 3 mPas that is 83% hydrolysed, i.e. having 83% of vinyl alcohol repeat units and 17% of vinyl acetate repeat units. A skilled person is aware that a hydrolysis grade of 83% and a viscosity of 3 mPas encompasses calculated hydrolysis grades of 82.50% to 83.49% and calculated viscosities of 2.50 mPas to 3.49 mPas % according to common rounding methods. Viscosity according to the invention is measured as stated in USP 39 under Monograph “Polyvinyl Alcohol” with the method Viscosity-Rotational Method (912).

The degree of hydrolysis according to the invention is measured by determining the saponification value of the Polyvinyl Alcohol, e.g. as stated in USP 39 under Monograph “Polyvinyl Alcohol” under “Degree of Hydrolysis”:
Sample: 1 g of Polyvinyl Alcohol, previously dried at 110° to constant weight

Analysis:

Transfer the Sample to a wide-mouth, 250-ml conical flask fitted by means of a suitable glass joint to a reflux condenser. Add 35 ml of dilute methanol (3 in 5), and mix gently to ensure complete wetting of the solid. Add 3 drops of phenolphthalein TS, and add 0.2 N hydrochloric acid or 0.2 N sodium hydroxide if necessary, to neutralize. Add 25.0 ml of 0.2 N sodium hydroxide VS, and reflux gently on a hot plate for 1 h. Wash the condenser with 10 ml of water, collecting the washings in the flask, cool, and titrate with 0.2 N hydrochloric acid VS. Concomitantly perform a blank determination in the same manner, using the same quantity of 0.2 N sodium hydroxide VS.

Calculation of saponification value:

Calculate the saponification value:

$$Result = [ ( {V}_{B} - {V}_{S} ) \times N \times {M}_{r} ] / W$$

V_{B}=volume of 0.2 N hydrochloric acid VS consumed in the titration of the blank (ml)
V_{S}=volume of 0.2 N hydrochloric acid VS consumed in the titration of the Sample solution (ml)
N=actual normality of hydrochloric acid VS
Mᵣ=molecular weight of potassium hydroxide, 56.11
W=weight of the portion of Polyvinyl Alcohol taken (g)

Calculation of degree of hydrolysis:

Calculate the degree of hydrolysis, expressed as a percentage of hydrolysis of polyvinyl acetate:

$$Result = 100 - [ 7.84 \times S / ( 100 - 0.075 \times S ) )$$

S=saponification value of the Polyvinyl Alcohol

The use of PVA grades according to the invention is of interest for the formulation of solid oral pharmaceutical dosage forms with an instant, immediate or prolonged API release.

Preferred PVAs have a hydrolysis degree of 70% to 90%, and a viscosity of a 4% solution at 20° C. of 3 mPas to 5 mPas, more preferably a viscosity of a 4% solution at 20° C. of 3 mPas to 4 mPas, most preferably a viscosity of a 4% solution at 20° C. of 4 mPas.

In a further embodiment of the invention, the polyvinyl alcohol has a hydrolysis degree of 70% to 90%, preferably 80% to 90% and a viscosity as mentioned above.

In a further embodiment of the invention, the polyvinyl alcohol has a hydrolysis degree of 80% to 90% and a viscosity of a 4% solution at 20° C. of 3 mPas or a hydrolysis degree of 80% to 90% and a viscosity of a 4% solution at 20° C. of 4 mPas.

In a further embodiment of the invention, the polyvinyl alcohol is PVA 3-80, PVA 3-82, PVA 4-88 or PVA 5-74, preferably PVA 3-80, PVA 3-82 or PVA 4-88, more preferably PVA 4-88.

Furthermore, surfactants can be added to the melt. Surfactants according to the present invention are compounds that absorb to surfaces or interfaces to reduce surface or interfacial tension. These agents usually act by reducing the interfacial tension between different surfaces. There are several general classes of surfactants used in pharmaceutical formulations. Exemplary anionic surfactants are Carboxylates (alkyl carboxylates-fatty acid salts), Sulfates (sodium lauryl sulfate, alkyl ether sulfates), Sulfonates (dioctyl sodium sulfosuccinate, alkyl benzene-sulfonates) or Phosphate esters (alkyl aryl ether phosphates, alkyl ether phosphates).

Exemplary cationic surfactants are Quaternary ammonium (cetrimonium bromide, cetylpyridinium chloride, dimethyldioctadecylammonium chloride), Amine-Based (triethylamine hydrochloride, octenidine dihydrochloride) or Pyridinium surfactants (benzethonium chloride).

Exemplary non-ionic surfactants are Polyol esters (fatty acid esters of sorbitan), Polyoxyethylene esters (polysorbates), Poloxamers (poloxamer 188), Amphoteric Phospholipids (phosphatidylcholine or lecithin), Carboxylic Acid/Quaternary Ammonium (cocamidopropyl betaine or amidosulfobetaine-16), Phosphoric Acid/Quaternary Ammonium (hexadecyl phosphocholine) or Betaines (alkylamidopropyl betaine).

In a further embodiment, exemplary suitable surfactants are Polyoxyethylene sorbitan fatty acid esters, (Polyoxyethylene 15 hydroxy stearate (Macrogol 15 hydroxy stearate, Solutol HS15®), Polyoxyethylene castor oil derivatives), Polyoxyethylene stearates, sorbitan fatty acid esters (Span®), Polyoxyethylene alkyl ethers (Brij®), and Polyoxyethylene nonylphenol ether (Nonoxynol®),

It has been surprisingly found that the addition of plasticizers enabled the modification of release rates and/or improved the surface finishing of the pharmaceutical dosage forms.

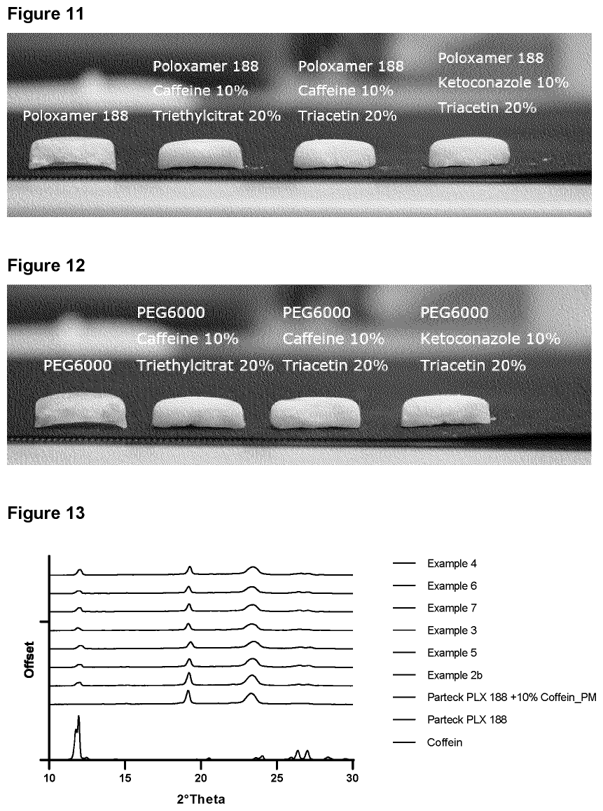

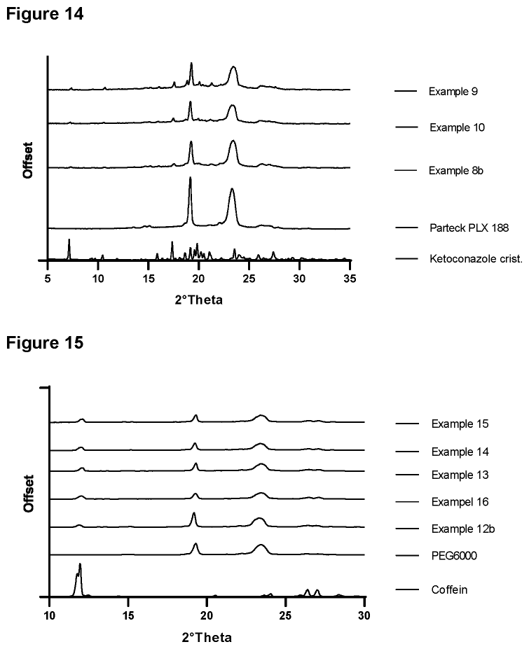

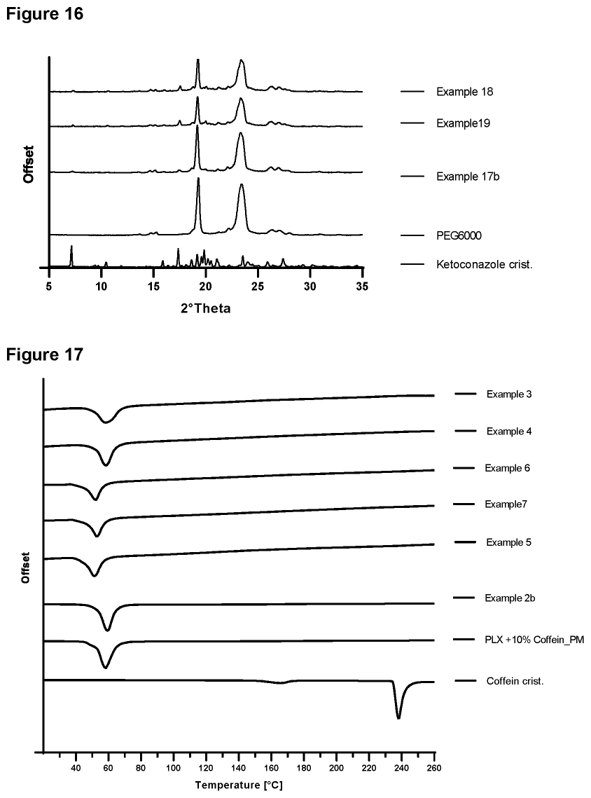

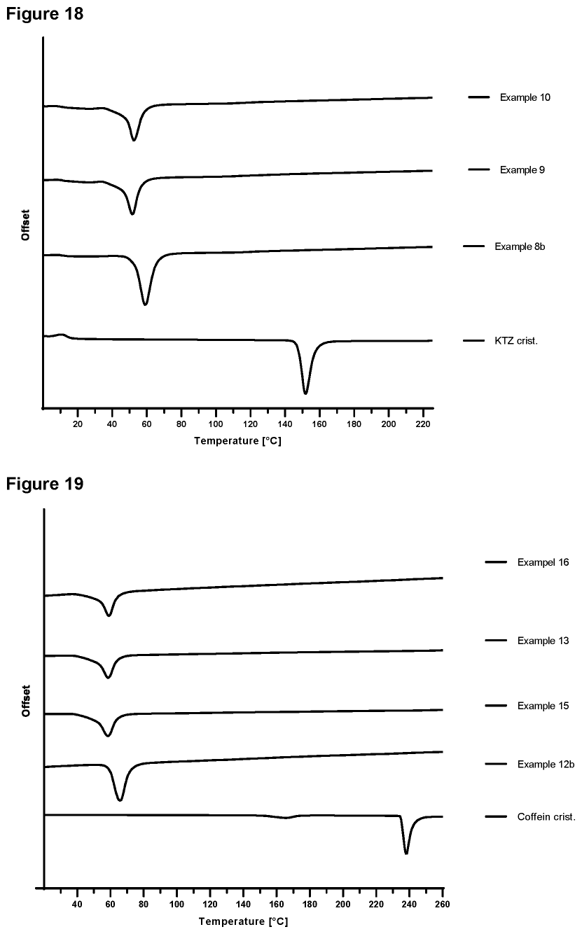

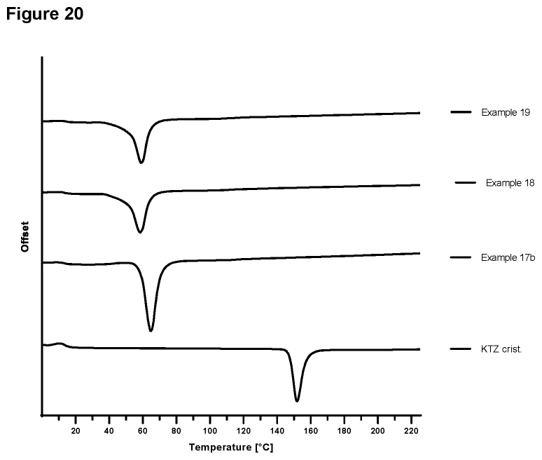

One particular challenge with the method of 3D melt dosing is the appearance of “edging” leading to a concave surface is created on top of the molds. As a consequence the surface finishing is negatively impacted by an uneven geometry which can be problematic for the final dose due to mechanical stability and optical appearance. The present invention shows that the edging can be minimized by adding plasticizers to the polymer. The effect can be seen in FIGS. 11 and 12 with triacetin and triethylcitrat as exemplary plasticizers. In these figures pharmaceutical dosage forms were turned upside down to have a better visibility of effect. Pharmaceutical dosage form comprising either triacetin or triethylcitrat have a less concave surface, preferably a flat surface.

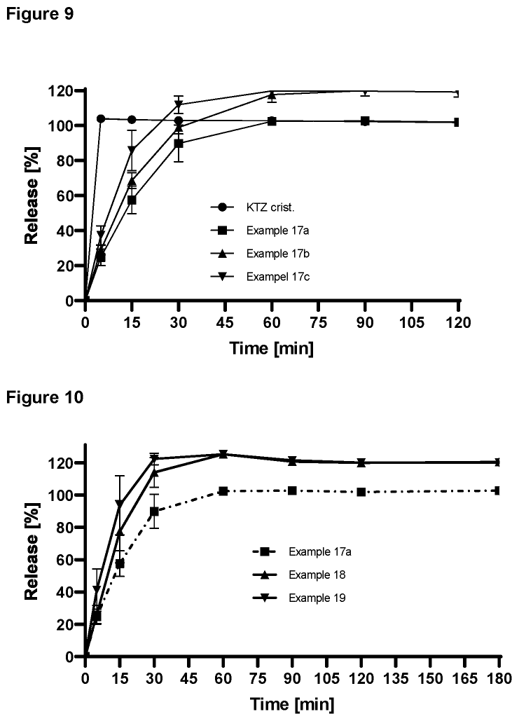

In a further aspect, drug release rates from pharmaceutical dosage forms manufactured by 3D melt dosing are rather prolonged because a relatively dense melting form is achieved. It was found that that the addition of the same plasticizers lead to an accelerated dissolution. In particular in combination with the polyethylene glycole polymer, triacetin and triethylcitrate the pharmaceutical dosage form show a faster drug release (FIG. 10).

In addition, using polymers with a relatively high melting point melt deposition technologies typically requires a reduction of the melting point, which can be obtained by mixing with excipients which have more suitable thermoplastic properties. A relatively low melting point excipient can give rise to plasticization if it is compatible with and mixed well with a polymeric carrier with a higher Tg. These plasticizers may either be small molecules or other polymers.

Examples of plasticizer include Glycerin, Acetyl Tributyl Citrate, Polyethylene Glycols, Acetyl Triethyl Citrate, Polyethylene Glycol, Monomethyl Ether, Castor Oil, Propylene Glycol, Diacetylated Monoglycerides, Sorbitol, Sorbitan, Solution Dibutyl Sebacate, Diethyl Phthalate, Triacetin, Tributylcitrate, Triethylcitrate or Polyols.

The API is a biologically active agent. The API may be a small molecule in form of a weak base, a weak acid or a neutral molecule and may be in the form of one or more pharmaceutically acceptable salts, esters, derivatives, analogues, prodrugs, and solvates thereof. The polymer and the pharmaceutical dosage form may comprise more than one API. In one embodiment the API is poorly soluble or a lipophilic API.

As used herein, the terms “poorly soluble API”, “poorly water-soluble API” and “lipophilic API” refer to an API having a solubility such that the highest therapeutic dose of the particular API to be administered to an individual cannot be dissolved in 250 ml of aqueous media ranging in pH from 1 to 8 following the definition of low solubility according to the Biopharmaceutics Classification System (BCS) classes 2 and 4. Poorly soluble APIs with weakly basic or weakly acidic characteristics have a pH-dependent solubility profile and can have a wide range of solubility in the aqueous environment of the gastrointestinal tract. APIs falling under BCS classes 2 or 4, respectively, are well known to persons skilled in the art.

In one embodiment the API is a weakly basic API. As used herein, the term “weakly basic API” refers to a basic active pharmaceutical ingredient (API) wherein the basic API does not completely ionize in water.

The at least one active pharmaceutical ingredient (API) according to the invention may be dispersed in the polymer forming an amorphous solid dispersion.

As used herein, the term “amorphous solid dispersion” is a dispersion of an amorphous API in a polymer matrix. Preferably, the amorphous API is distributed in a molecularly dispersed state within the polymer matrix. In this case, the solid dispersion is a solid solution. Upon dissolution, formulations comprising an amorphous solid dispersion can reach higher solubilities in aqueous media than the crystalline API.

The API included in the pharmaceutical dosage form of the present invention has a sufficient amount to be therapeutically effective. For a given API, therapeutically effective amounts are generally known or readily accessible by persons skilled in the art. Typically, the API may be present in the pharmaceutical dosage form in a weight ratio of API to polyvinyl alcohol of 0.1:99.1 to 60:40, preferably 1:99 to 50:50, more preferably 5:95 to 40:60 and most preferably 10:90 to 30:70.

A further embodiment of the invention is a pharmaceutical dosage form obtainable by the process for manufacturing of a pharmaceutical dosage form with 3D melt dosing as defined above.

### EXAMPLES

### Example 1

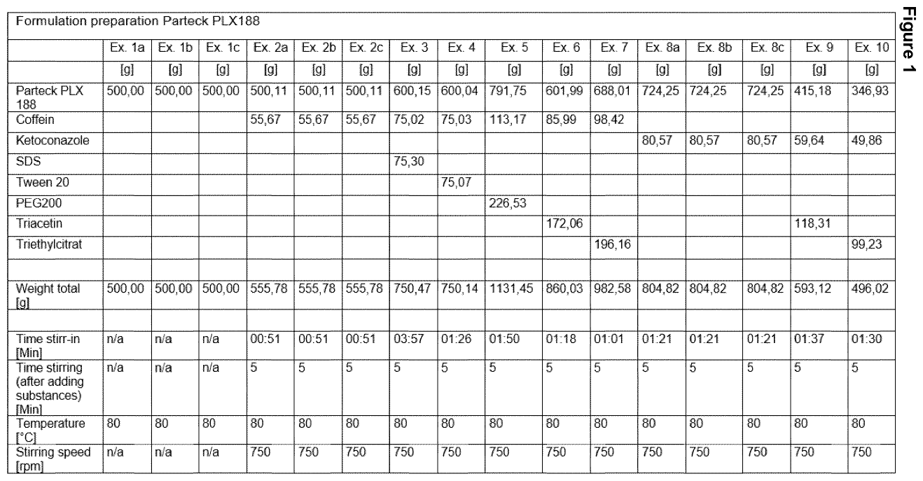

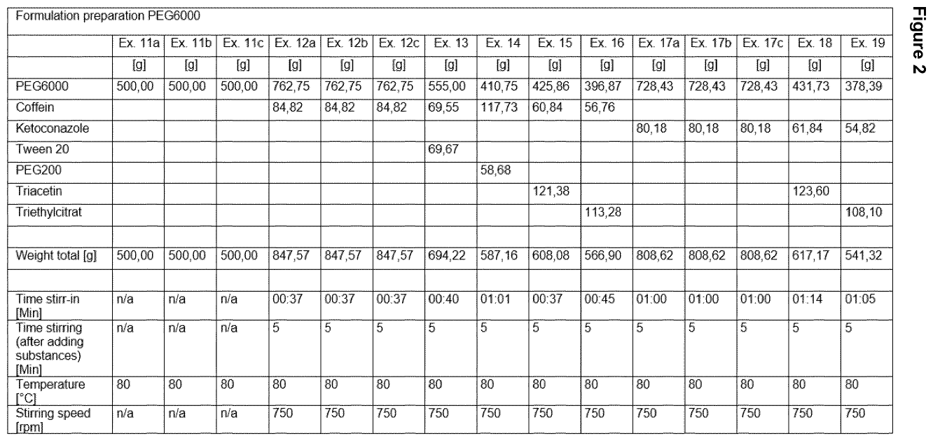

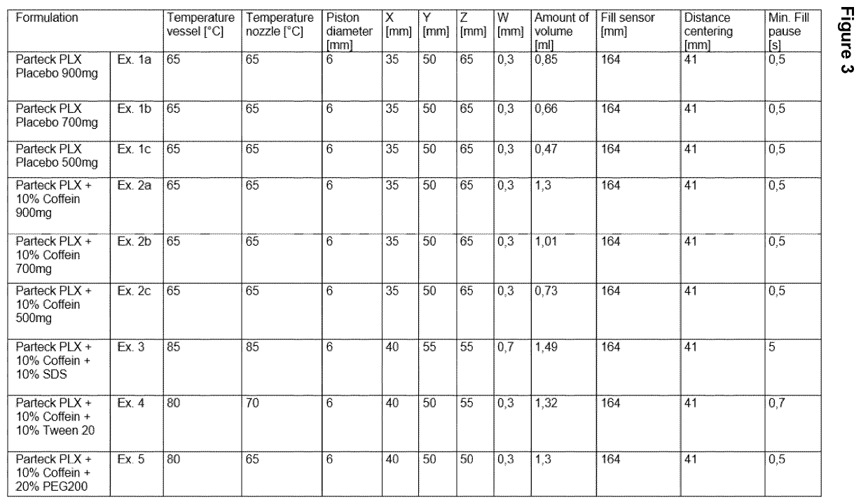

Formulations 1 to 19 (Examples 1 to 19) were prepared with either Poloxamer 188 (Parteck PLX 188) or PEG6000 as polymer. In some formulations APIs (Coffein or Ketokonazole) or additives (SDS, Tween 20, PEG200, Triacetin or Triethylcitrate) were added. The exact masses and stir times can be found in FIG. 1 and FIG. 2.

Formulations were prepared by
1. Melting the polymer in a drying chamber at 80° C.,
2. Weighing the melted material and stirring in a beaker on a hot plate (80° C.) at 750 rpm to create a vortex,
3. Weighing the exact amount of API (10%) and/or additives (10% or 20%) respectively and dispersing in the melt,
4. Stirring-in the API and/or additive with a paddle stirrer (see FIGS. 1 and 2 for stir-in time),
5. Stirring for 5 minutes.

Tablet formulation was performed with a KCM Omega Sep. 18, 1935 machine (Knobel). The first step of the procedure is transferring the melt from the beaker to the machine vessel. The tray which contains the moulding form is put onto a running conveyor in the right position. The conveyor transports the tray further until it reaches the filling sensor. The filling sensor is a parameter, which can be adjusted in the software, so that the tray stops precisely under the nozzle. Meanwhile, the nozzle moves to position Z. During this step, the pumps fill with a specified amount of volume. The nozzle lower itself to position X (near the ground of the form) and the piston turns and start to discharge the melt. The nozzle remains in this position during the wait delay W. After that, the nozzle moves vertically upwards to the Y position, while the piston continue to discharge the melt. After discharging the melt, the piston rotate to the suction position and the nozzle ascend back to position Z. The conveyor moves the tray to the next mould after the fill pause is expired. The suction cycle of the piston is also performed simultaneously. The right distance between each form can be adjusted with the distance centering button.

This work procedure repeats itself until all desired number of forms are filled. The tray moves then to the stop sensor and the production process is finished.

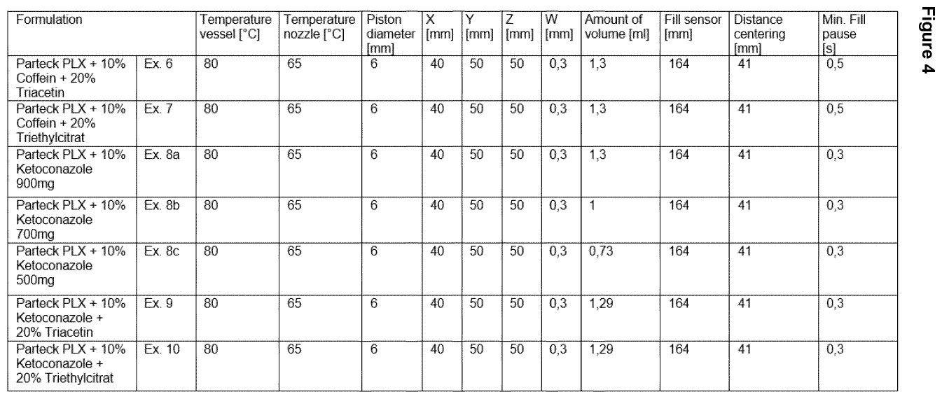

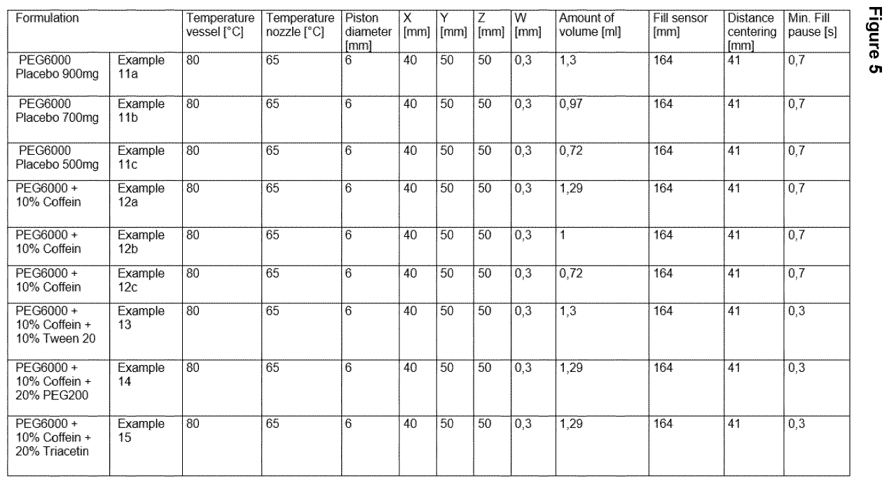

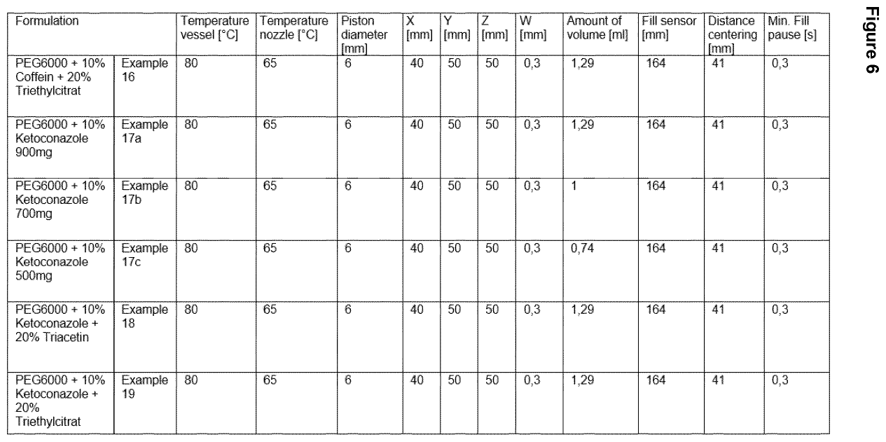

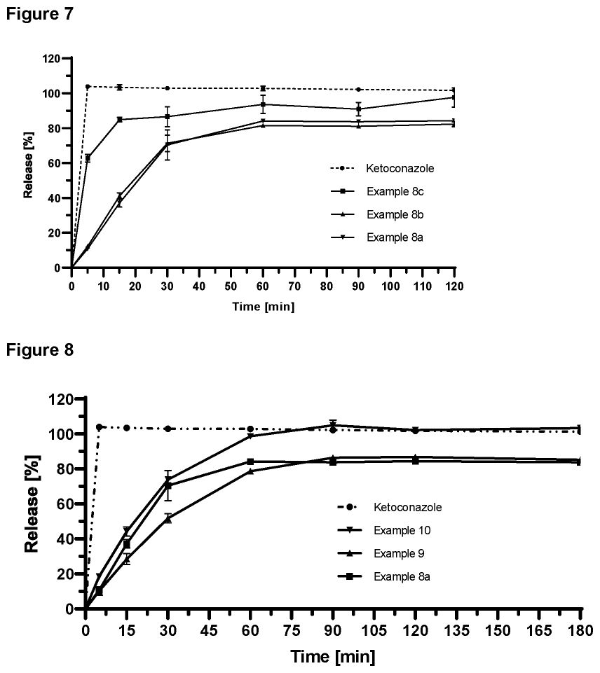

The detailed machine settings for each formulation can be seen in FIG. 3 to FIG. 6.

### Example 2

Dissolution performance was measured on a Sotax AT7 smart with a fraction collector with the following conditions:
Dissolution medium: 900 ml 0.1 M HCL, 37° C.
50 rpm; Paddle; prefilter glass Microfiber Filters GE Whatman GF/D Diameter 25 mm
Sampling Points: 5, 15, 30, 60, 90, 120, 180
Sampling Volume: 2.5 ml volume was not added after sampling

Sample preparation HPLC (Ketoconazole):

All samples were filtered with a PTFE 0.45 μm Filter (VWR 13 mm Syringe Filter/0.45 μm PTFE Membrane) and diluted with Ethanol

HPLC conditions:
Mobile Phase Ketoconazole is composed of two Eluents.
Eluent A: 10 ml Diisopropylamine and 5 L Methanol.
Eluent B: 25 g Ammoniumacetat and 5 L MilliQ water
Ratio: 70% Eluent A: 30% Eluent B; isocratic
Column: SUPELCOSIL LC-18-18, 5 μm 300 mm×4 mm
HPLC Agilent 1260 Infinity or 1260 Infinity II with UV detector,
Parameter: Runtime: 8 min; Flow: 2 ml; 252 nm; injection volume 10 μl; 40° C. column oven
Retention Time Peak: 6.5 Min

### Example 3

Used friability testing device: ERWEKA TA 420 (ERWEKA GmbH, Heusenstamm, Germany). The test method was performed accorting to Ph. EUR. EP 10.7, paragraph 2.9.7 Friability of uncoated tablets.

For tablets with a unit mass equal to or less than 650 mg, take a sample of whole tablets corresponding as near as possible to 6.5 g. For tablets with a unit mass of more than 650 mg, take a sample of 10 whole tablets. The tablets are carefully dedusted prior to testing. Accurately weigh the tablet sample, and place the tablets in the drum. Rotate the drum 100 times, and remove the tablets. Remove any loose dust from the tablets as before, and accurately weigh.

Implementation: The tablets were dusted, and their weight (m0) determined. The drum rotated 100 times; after that, the tablets were again dusted and weighed (m1). Abrasion should be less than 1% (Ph. Eur.).

$$Calculation : Abrieb [ \% ] = ( 1 - ( \frac{m 1}{m 0} ) ) \times 100$$

Physical characterization of tablets via Erweka Multicheck 5.1 (ERWEKA GmbH, Heusenstamm, Germany), with his automated device tablet dimensions, hardness as well as tablet weight can be determined. For each setup 20 tabletes were randomly selected and the parameters were determined.

The homogeneity of the surface can be improved by the addition of triethylcitrate and triacetin plasticizers (FIGS. 11 and 12). This is an important aspect to further optimize the finishing of the dosage forms. It was found that especially can improve the surface finishing and enable a homogenous manufacturing.

### Example 4

Device: MiniFlex 300/600 Rigaku Corporation (Tokyo/Japan)
For the measurement, the tablets were milled with a Tube Mill 100 (IKA-Werke GmbH&CO KG) and sieved with an 250 μm sieve. Samples were measured in transmission mode at 40 kV and 15 mA with a velocity of 10°/minute within a range of 3°-60°. Copper was used as an anode material at a wavelength of 1.54060 A. Stepsize: 0.02°.

Traces of crystalline Caffeine are still observed also in the provided examples (12° 2Theta and around 26 & 27° 2Theta).

Also for ketoconazole traces of crystalline forms can be observed at 7 and 17° 2Theta. Device: DSC 3+ (Mettler Toledo)

The milled tablets were weighed in a 40 μl aluminum pan. The weighed portion for each sample was between 5 mg to 6 mg. A temperature profile from −25° C. to 230° C. (Ketoconazole) and −25° C. to 260° C. (Caffeine) with a heat flow of 30K/minute was selected. Nitrogen gas was applied with a flow rate of 50 ml/minute.

Thermal analysis indicate a full amorphization of the drug substance, but effects are rather linked to the process related melting of the polymer during heating at about 60° C.

Thermal analysis indicate a full amorphization of the drug substance, but effects are rather linked to the process related melting of the polymer during heating at about 60° C.-70° C.

1. A pharmaceutical dosage form, wherein the pharmaceutical dosage form is manufactured using a polymer in a 3D melt dosing process, wherein the polymer has a melting point between 20° C. and 200° C.

2. The pharmaceutical dosage form according to claim 1, wherein the polymer is semi-crystalline.

3. The pharmaceutical dosage form according to claim 1, wherein the polymer comprises a plasticizer.

4. The pharmaceutical dosage form according to claim 1, wherein the polymer has a melting point between 20° C. and 80° C.

5. The pharmaceutical dosage form according to claim 1, wherein the polymer is a poloxamer or polyethylene glycol.

6. Process for manufacturing of a pharmaceutical dosage form with 3D melt dosing, comprising the steps of
1. melting a polymer with a melting point between 20° C. and 200° C.,
2. adding at least one active pharmaceutical ingredient to the molden polymer and
3. dispensing the mixture through a first nozzle into a mould.

7. Process according to claim 6, wherein a second polymer is simultaneously or sequentially dispensed through the nozzle into the mould.

8. Process according to claim 6, wherein a second polymer is simultaneously or sequentially dispensed through a second nozzle into the mould.

9. Process according to claim 6, wherein the polymer is semi-crystalline.

10. Process according to claim 6, wherein a plasticizer is added to the molden polymer.

11. Process according to claim 6, wherein the polymer has a melting point between 20° C. and 80° C.

12. Process according to claim 6, wherein the polymer is a poloxamer or polyethylene glycol.

13. A pharmaceutical dosage form obtainable by a process according to claim 6.
